(19) United States
(12) Patent Application Publication
(10) Pub. No.: US 2014/0161837 A1
(43) Pub. Date: Jun. 12, 2014

(54) VACCINE ADJUVANT, VACCINE COMPOSITION AND METHOD FOR PREPARING A VACCINE ADJUVANT

(71) Applicant: Industrial Technology Research Institute, Hsinchu (TW)

(72) Inventors: I-Horng PAN, Hsinchu City (TW); I-Haung LU, Taipei City (TW); Hsin-Jan YAO, Yunlin County (TW); Hsin-Chieh WU, Hsinchu City (TW); Chi-Chien LIN, Nantou City (TW)

(73) Assignee: Industrial Technology Research Institute, Hsinchu (TW)

(21) Appl. No.: 14/101,044

(22) Filed: Dec. 9, 2013

(30) Foreign Application Priority Data
Dec. 11, 2012 (TW) 101146538

(51) Int. Cl. A61K 39/00; C08B 37/00

(52) U.S. Cl. CPC A61K 39/0002; A61K 39/0011; C08B 37/0003
USPC 424/204.1; 424/234.1; 424/269.1; 424/274.1; 536/123.1

(57) ABSTRACT

The disclosure provides a vaccine adjuvant, including a polysaccharide derived from Antrodia camphorata (also named Antrodia cinnamomea or Taiwanofungus camphoratus) fruiting body, wherein the molecular weight of the polysaccharide is greater than 100 K Da. Furthermore, the polysaccharide is obtained by an extraction process, and the extraction process includes: (a) adding powder of the Antrodia camphorata fruiting body into water to form a mixture; (b) heating the mixture under reflux; (c) after step (b), removing an insoluble matter from the mixture; (d) after step (c), adding ethanol into the mixture to perform a precipitating step and obtain a precipitate; and (e) performing an isolating step to the precipitate to obtain a fraction the molecular weight of which is greater than 100 K Da of the precipitate.

### CROSS REFERENCE TO RELATED APPLICATIONS

This application claims priority of Taiwan Patent Application No. 101146538, filed on Dec. 11, 2012, the entirety of which is incorporated by reference herein.

### TECHNICAL FIELD

The technical field relates to a vaccine adjuvant, a vaccine composition and a method for preparing a vaccine adjuvant.

### BACKGROUND

Vaccine is capable of starting a humoral immune response and then producing antibodies, or activating lymphocytes, such as cytotoxic T cells through a cellular immune response to resist the invasion of a pathogenic organism and prevent occurrence of disease (Cavallo F et al., Vaccination for treatment and prevention of cancer in animal models. Adv Immunol. 2006. 90:175-213. Review). Although vaccines have the effect of activating a subject's immune system, in clinical use, it is often found that the vaccine cannot perform the desired effect in some populations whose auto-immune systems are too weak, such as the aged and children, and thus the addition of the proper amount of vaccine adjuvant is needed. Furthermore, addition of a vaccine adjuvant also has the effect of promoting the immune system to recognize an antigen, and the antigen can be more effectively used through promoting the immune response to decrease the vaccine dosage and vaccine frequency. Therefore, the addition of a vaccine adjuvant not only can decrease the cost of the vaccine, but it can also increase the immune efficiency of the vaccine.

According to the functions of adjuvants, adjuvants can be classified into two groups. Adjuvants belonging to the first group are used for absorbing antigens and assisting antigens to be phagocytized by cells, such as aluminum salts and M59 emulsifying agent, etc. (O'Hagan D T, Wack A, Podda A. MF59 is a safe and potent vaccine adjuvant for flu vaccines in humans: what did we learn during its development? Clin Pharmacol Ther. 2007 December; 82(6):740-4; 4. Clapp T, Siebert P, Chen D, Jones Braun L. Vaccines with aluminum-containing adjuvants: optimizing vaccine efficacy and thermal stability. J Pharm Sci. 2011 February; 100(2):388-401); adjuvants belonging to the other group are immune regulatory factors, such as CFA-mycobacteria, etc. (Hoft D F, Blazevic A, Abate G, Hanekom W A, Kaplan G, Soler J H, Weichold F, Geiter L, Sadoff J C, Horwitz M A. A new recombinant bacille Calmette-Guérin vaccine safely induces significantly enhanced tuberculosis-specific immunity in human volunteers. J Infect Dis. 2008 Nov. 15; 198(10):1491-501). The main function of a vaccine adjuvant is enhancing the immune activity of an antigen and the immune protective effect, however it has been confirmed that common aluminum salt adjuvants have selectivity for vaccines. Accordingly, the development of a new vaccine adjuvant is needed to promote antigen specificity of the vaccine or the anti-tumor and anti-infection ability of the vaccine.

### SUMMARY

The disclosure provides a vaccine adjuvant, comprising: a polysaccharide derived from Antrodia camphorata (also named Antrodia cinnamomea or Taiwanofungus camphoratus) fruiting body, wherein the molecular weight of the polysaccharide is greater than 100 K Da.

The disclosure also provides a vaccine composition, comprising: the vaccine adjuvant as mentioned above; and an antigen or DNA encoding the antigen.

The disclosure further provides a method for preparing a vaccine adjuvant, using a polysaccharide derived from Antrodia camphorata (also named Antrodia cinnamomea or Taiwanofungus camphoratus) fruiting body.

A detailed description is given in the following embodiments with reference to the accompanying drawings.

### BRIEF DESCRIPTION OF THE DRAWINGS

The embodiments of disclosure can be more fully understood by reading the subsequent detailed description and examples with references made to the accompanying drawings, wherein:

FIG. 1A shows the preparation process for the polysaccharide from the Antrodia camphorata fruiting body (ACFB01) sample;

FIG. 1B shows the preparation process for the polysaccharide from the Antrodia camphorata fruiting body (ACFB01>100 K) sample;

FIG. 2 shows the gel filtration chromatography profile for ACFB01>100 K sample;

FIG. 3 shows the levels of TNF-α secreted by mouse bone marrow-derived dendritic cells (BMDCs) treated with the polysaccharide of Antrodia camphorata fruiting body, ACFB01 sample, at different doses, and cell viability;

FIG. 4A shows the levels of TNF-α secreted by mouse bone marrow-derived dendritic cells (BMDCs) treated with fractions with different molecular weight of ACFB01 sample;

FIG. 4B shows the levels of TNF-α secreted by mouse bone marrow-derived dendritic cells (BMDCs) treated with ACFB01>100 K sample at different doses;

FIGS. 5A and 5B show the levels of IL-6 and IL-12 secreted by mouse bone marrow-derived dendritic cells (BMDCs) treated with ACFB01>100 K sample at different doses (0-20 μg/ml);

FIGS. 6A, 6B and 6C show the levels of MCP-1, MIP-1α and RANTES secreted by mouse bone marrow-derived dendritic cells (BMDCs) treated with ACFB01>100 K sample at different doses (0-20 μg/ml), respectively;

FIGS. 7A, 7B and 7C show expression conditions of CD40, CD86 and MHC class II of mouse bone marrow-derived dendritic cells (BMDCs) treated with ACFB01>100 K sample (20 μg/ml), respectively;

FIG. 8A shows the effect of ACFB01>100 K sample on T cell proliferation in vitro;

FIG. 8B shows the effect of ACFB01>100 K sample on expression levels of Interferon-γ/IFN-γ and IL-4 in vitro;

FIG. 9A shows the effect of ACFB01>100 K sample on T cell proliferation in vivo;

FIG. 9B shows the effect of ACFB01>100 K sample on expression levels of Interferon-γ/IFN-γ and IL-4 in vivo;

FIG. 10A shows the effect of ACFB01>100 K combined with HER-2/neu DNA vaccine on the tumor of C3/HeN mice injected with MBT-2 tumor cells (bladder cancer cell overexpressing HER-2/neu);

FIG. 10B shows the effect of ACFB01>100 K combined with HER-2/neu DNA vaccine on the life of C3/HeN mice injected with MBT-2 tumor cells (bladder cancer cell overexpressing HER-2/neu);

FIGS. 11A and 11B show the effect of ACFB01>100 K combined with HER-2/neu DNA vaccine on activation of T cells of C3/HeN mice injected with MBT-2 tumor cells (bladder cancer cell overexpressing HER-2/neu);

FIG. 11C shows the effect of ACFB01>100 K combined with HER-2/neu DNA vaccine on expressions of IFN-γ and IL-4 of C3/HeN mice injected with MBT-2 tumor cells (bladder cancer cell overexpressing HER-2/neu); and

FIG. 12 shows the effect of dendritic cell vaccine pulsed with ACFB01>100 K treatment on tumor of the orthotopic liver cancer model.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In one embodiment, the present disclosure provides a vaccine adjuvant which comprises a polysaccharide derived from Antrodia camphorata fruiting body.

The molecular weight of the polysaccharide derived from Antrodia camphorata fruiting body mentioned above may be greater than 100 K Da. For example, the molecular weight of the polysaccharide mentioned above may be between 2.0×10⁵ Da and 2.1×10⁷ Da, but is not limited thereto. In addition, in one embodiment, the polysaccharide derived from Antrodia camphorata fruiting body mentioned above may comprise, but is not limited to, a part of which the molecular weight is about 2.4×10⁵˜2.5×10⁵ Da, a part of which the molecular weight is about 2.4×10⁶˜2.5×10⁶ Da, a part of which the molecular weight is about 1.0×10⁷˜1.1×10⁷ Da, and a part of which the molecular weight is about 2.0×10⁷˜2.1×10⁷ Da.

The polysaccharide derived from Antrodia camphorata fruiting body of which the molecular weight may be greater than 100 K Da may be obtained by an extraction process. In one embodiment, the extraction process may comprise the following steps, but is not limited thereto.

First, powder of the Antrodia camphorata fruiting body is added to water to form a mixture.

Next, a step of heating under reflux is performed to the foregoing mixture. In one embodiment, the time for heating under reflux is about 1-3 hours. In another embodiment, the time for heating under reflux may be about 1 hour.

Then, after the preceding step of heating under reflux, an insoluble matter is removed from the mixture. In one embodiment, an insoluble matter is removed from the mixture through a filtering step.

After removing the insoluble matter from the mixture, ethanol is added to the mixture to perform a precipitating step and obtain a precipitate. In one embodiment, the ethanol may comprise 95% ethanol. Moreover, in one embodiment, the time for precipitating may be about 8-24 hours.

Finally, an isolating step is performed to the precipitate to obtain a fraction of the precipitate, the molecular weight of which is greater than 100 K Da. In one embodiment, the isolating step is performed by an apparatus which can isolate an ingredient that has a specific molecular weight, such as the Amicon® Ultra Centrifugal Filter Device (UFC9 100 08: 15 mL, 100K NMWL, MILLPORE), but it is not limited thereto.

In one embodiment, the polysaccharide derived from Antrodia camphorata fruiting body of which the molecular weight may be greater than 100 K may be dispersed in an aqueous solution, and then the aqueous solution can be homogeneously emulsified with an emulsifying agent and an oil to form the vaccine adjuvant of the present disclosure.

The polysaccharide derived from Antrodia camphorata fruiting body of which the molecular weight may be greater than 100 K mentioned above may be capable of activating a dendritic cell.

Furthermore, the polysaccharide derived from Antrodia camphorata fruiting body of which the molecular weight may be greater than 100 K mentioned above may be capable of enhancing a dendritic cell to express a major histocompatibility complex (MHC) class II, CD40 and/or CD86.

In addition, the polysaccharide derived from Antrodia camphorata fruiting body of which the molecular weight may be greater than 100 K mentioned above may be capable of enhancing a dendritic cell to induce activation of an antigen-specific T cell.

The polysaccharide derived from Antrodia camphorata fruiting body of which the molecular weight may be greater than 100 K mentioned above may be capable of enhancing T cell proliferation and/or expression of interferon which is a Th1 cell cytokine.

In one embodiment, the vaccine adjuvant of the present disclosure may be mixed with an antigen to form a vaccine composition. Examples for suitable antigens may comprise phage, phage composition, virus, virus composition, rickettsia, rickettsia composition, actinomyces, actinomyces composition, bacteria, bacteria composition, fungus, fungus composition, protozoan, protozoan composition, tumor tissue, tumor cell, tumor cell composition, tumor antigen protein, and tumor antigen peptide, etc., but it is not limited thereto.

In one embodiment, the content of the vaccine adjuvant in the vaccine composition is about 10-50 wt %.

Furthermore, in one embodiment, the vaccine adjuvant of the present disclosure may be combined with a vaccine and used. The vaccine mentioned above may comprise, but is not limited to, an anti-cancer vaccine, an anti-virus vaccine, or an anti-bacteria vaccine.

In one embodiment, the vaccine may be an anti-cancer vaccine. In this embodiment, a cancer which can be protected against by the preceding anti-cancer vaccine may comprise, but is not limited to, bladder cancer, liver cancer, leukemia, colorectal cancer, breast cancer, kidney cancer, lung cancer, pancreatic cancer, prostate cancer, cervical cancer, or head and neck cancer, etc.

Moreover, in one embodiment, the preceding anti-cancer vaccine may comprise a DNA vaccine or a dendritic cell (DC) vaccine, but it is not limited thereto.

In another embodiment of the present disclosure, the present disclosure also provides a vaccine composition which comprises the vaccine adjuvant of the present disclosure mentioned above and an antigen or DNA encoding the antigen. In one embodiment, the content of the vaccine adjuvant in the vaccine composition mentioned above is about 10-50 wt %. Moreover, in one embodiment, the content of the antigen in the vaccine composition mentioned above is about 50-90 wt %.

In one embodiment, in the vaccine composition, the molecular weight of the polysaccharide derived from Antrodia camphorata fruiting body is greater than 100 K Da, more specifically, the molecular weight of the polysaccharide derived from Antrodia camphorata fruiting body is between 2.0×10⁵ Da and 2.1×10⁷ Da.

Furthermore, in the vaccine composition, the antigen may comprise, but is not limited to, phage, phage composition, virus, virus composition, rickettsia, rickettsia composition, actinomyces, actinomyces composition, bacteria, bacteria composition, fungus, fungus composition, protozoan, protozoan composition, tumor tissue, tumor cell, tumor cell composition, tumor antigen protein, or tumor antigen peptide, etc.

The type of the vaccine composition of the present disclosure may comprise an anti-cancer vaccine composition, an anti-virus vaccine composition, or an anti-bacteria vaccine composition, but is not limited thereto.

In one embodiment, the vaccine composition of the present disclosure may be an anti-cancer vaccine composition. The preceding anti-cancer vaccine composition can be used against bladder cancer, liver cancer, leukemia, colorectal cancer, breast cancer, kidney cancer, lung cancer, pancreatic cancer, prostate cancer, cervical cancer or head and neck cancer, etc., but it is not limited thereto. In addition, the preceding anti-cancer vaccine composition may comprise, but is not limited to, a DNA vaccine composition or a dendritic cell (DC) vaccine composition.

Moreover, in yet another embodiment, the present disclosure further provides a method for preparing a vaccine adjuvant, wherein the method comprises using a polysaccharide derived from Antrodia camphorata fruiting body. The molecular weight of the polysaccharide derived from Antrodia camphorata fruiting body mentioned above may be greater than 100 K Da. For example, the molecular weight of the polysaccharide mentioned above may be between 2.0×10⁵ Da and 2.1×10⁷ Da, but is not limited thereto. In one embodiment, the polysaccharide derived from Antrodia camphorata fruiting body mentioned above may comprise, but is not limited to, a part of which the molecular weight is about 2.4×10⁵˜2.5×10⁵ Da, a part of which the molecular weight is about 2.4×10⁶˜2.5×10⁶ Da, a part of which the molecular weight is about 1.0×10⁷˜1.1×10⁷ Da, and a part of which the molecular weight is about 2.0×10⁷˜2.1×10⁷ Da.

The polysaccharide derived from Antrodia camphorata fruiting body of which the molecular weight may be greater than 100 K Da may be obtained by an extraction process. In one embodiment, the extraction process may comprise the following steps, but it is not limited thereto.

First, powder of the Antrodia camphorata fruiting body is added to water to form a mixture.

Next, a step of heating under reflux is performed to the foregoing mixture. In one embodiment, the time for heating under reflux is about 1-3 hours. In another embodiment, the time for heating under reflux may be about 1 hour.

After the preceding step of heating under reflux, an insoluble matter is removed from the mixture. In one embodiment, an insoluble matter is removed from the mixture through a filtering step.

After removing the insoluble matter from the mixture, ethanol is added to the mixture to perform a precipitating step and obtain a precipitate. In one embodiment, the ethanol may comprise 95% ethanol. Moreover, in one embodiment, the time for precipitating may be about 8-24 hours.

Finally, an isolating step is performed to the precipitate to obtain a fraction the molecular weight of which is greater than 100 K Da of the precipitate. In one embodiment, the isolating step is performed by an apparatus which is capable of isolating an ingredient that has a specific molecular weight, such as Amicon® Ultra Centrifugal Filter Device (UFC9 100 08: 15 mL, 100K NMWL, MILLPORE), but it is not limited thereto.

In one embodiment, the foregoing vaccine adjuvant may be combined with a vaccine and used. The vaccine mentioned herein may comprise, but is not limited to, an anti-cancer vaccine, an anti-virus vaccine or an anti-bacteria vaccine.

In one embodiment, the vaccine mentioned above may be an anti-cancer vaccine. In this embodiment, a cancer which can be protected against by the preceding anti-cancer vaccine may comprise, but is not limited to, bladder cancer, liver cancer, leukemia, colorectal cancer, breast cancer, kidney cancer, lung cancer, pancreatic cancer, prostate cancer, cervical cancer, or head and neck cancer, etc.

Moreover, in one embodiment, the preceding anti-cancer vaccine may comprise a DNA vaccine or a dendritic cell (DC) vaccine, but it is not limited thereto.

### Examples

### 1. Preparation of Polysaccharide Derived from the Antrodia camphorata Fruiting Body

A. Preparation of Sample of Crude Polysaccharide Derived from the Antrodia camphorata fruiting body (ACFB01).

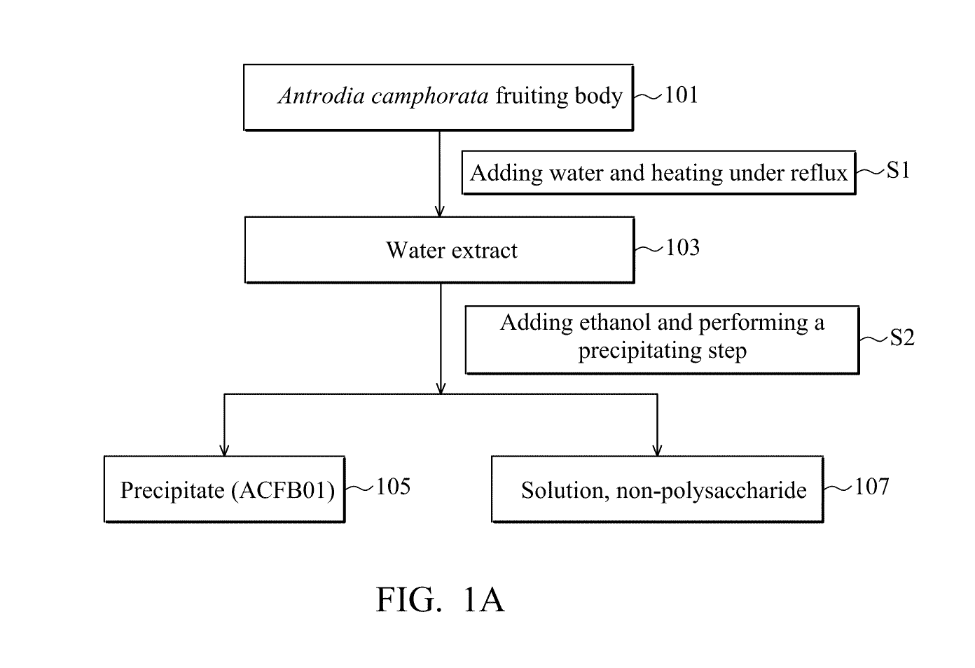

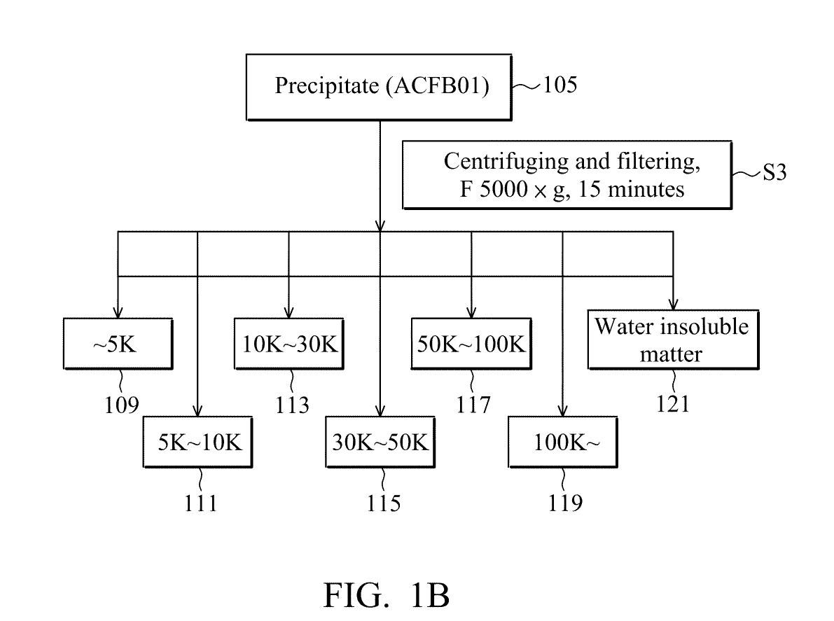

First, a crude polysaccharide was extracted from the Antrodia camphorata, and the extraction process is shown in FIG. 1A, wherein the detailed process is described in the following.

(1) 600 g of Antrodia camphorata fruiting body was pulverized, and then added to 2400 ml pure water to form a mixture and heated under reflux for 1 hour (Step S1).

(2) The insoluble matter was filtered out from the mixture by pressure reducing filtration while the mixture was still hot (Step S2).

(3) Steps (1)-(2) were repeated to the insoluble matter, and the three rounds of filtrates which were obtained from the preceding steps were combined, wherein a total of 6.254 kg filtrate was obtained.

(4) The filtrate was slowly added to 4-fold amount of 95% ethanol with a total of 25 Kg (50 ml/minute), and stirred with a paddle (25 rpm/minute) to be mixed (Step S3).

(5) After the filtrate was completely added to the 95% ethanol, the obtained solution stood for 24 hours.

(6) The supernatant was sucked out, and the bottom containing a precipitate was centrifuged (3000×g, 15 minutes) to remove the remaining solution.

(7) The precipitate was placed in a suction container for 1 hour, and after the ethanol was completely vaporized, the precipitate was lyophilized to remove the remaining water.

(8) A total of 11.73 g lyophilized product was collected, and this product was crude polysaccharide, named ACFB01.

B. Preparation of Sample of Polysaccharide of Antrodia camphorata Fruiting Body (ACFB01>100 K).

A further isolating process was performed to the crude polysaccharide mentioned above, and the isolating process is shown in FIG. 1B, wherein the detailed process is described in the following.

(1) 2.0 g of the crude polysaccharide obtained above was added to a 10-fold amount of pure water (20 g) to form a mixture, and heated to 90° C. for 1 hour.

(2) The mixture was centrifuged (3000×g) for 15 minutes to remove the precipitate.

(3) The supernatant was placed in an inner column of Amicon® Ultra Centrifugal Filter Device (UFC9 100 08: 15 mL, 100K NMWL, MILLPORE) and centrifuged (5000×g) for 15 minutes, and then the liquids in the inner column and outer column were collected, separately (Step S4).

(4) 10 mL pure water was added to the liquid from the inner column and mixed well (vortexed), and then step (3) was repeated.

(5) Steps (3)-(4) were repeated 3 times, and the liquid from the inner column was collected and frozen by liquid nitrogen, and then lyophilized. The obtained product was a fraction of the polysaccharide, a molecular weight of which is greater than 100 K Da, named ACFB01>100 K.

(6) The liquid from the outer column of step (5) was collected, and by using different catalog numbers (different fraction) of Amicon® Ultra Centrifugal Filter Device, polysaccharides differentiated by different molecular weights could be further obtained from the collected liquid from the outer column. The weight and the weight percentage of the polysaccharides with different molecular weights in the crude polysaccharide are shown in Table 1.

TABLE 1
Weight and the weight percentage of the polysaccharides with
different molecular weights in the crude polysaccharide.

| | Molecular weight (Da)/Isolated matter | | | | | | |
|---|---|---|---|---|---|---|---|
| | | | | | | | Water |
| | | | | | | | insoluble |
| | ~5K | 5K~10K | 10K~30K | 30K~50K | 50K~100K | 100K~ | matter |
| Weight | 229.9 mg | 47.3 mg | 155.7 mg | 212.9 mg | 308.0 mg | 915.9 mg | 96.3 mg |
| Weight | 11.69% | 2.40% | 7.92% | 10.83% | 15.67% | 46.59% | 4.90% |
| percentage | | | | | | | |

### 2. Gel Filtration Chromatography Profile for Polysaccharide Contained by the ACFB01>100 K Sample and the Composition of the Polysaccharide Contained by the ACFB01>100 K Sample

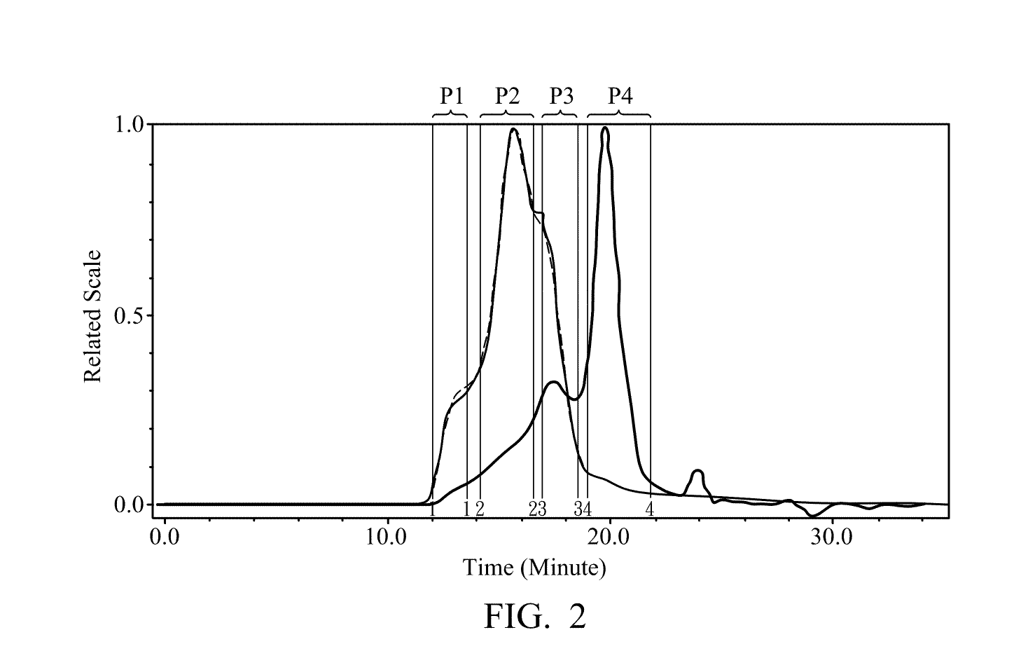

The molecular weight distribution for ACFB01>100 K sample and the weight ratios of different molecular weight parts in ACFB01>100 K sample were determine by high performance liquid chromatography (HPLC) combined with multi-angle laser light scatter, UV and RI detectors. The result shows 4 regions which represent mean molecular weights of about 2.4×10⁵˜2.5×10⁵ Da, about 2.4×10⁶˜2.5×10⁶ Da, about 1.0×10⁷˜1.1×10⁷ Da and about 2.0×10⁷˜2.1×10⁷ Da, respectively (FIG. 2). According to the above mentioned, it is understood that molecular weight distribution for ACFB01>100 K sample is about 2.0×10⁵ Da and 2.1×10⁷ Da. The weight ratios of the four regions in ACFB01>100 K sample are shown in Table 2.

TABLE 2
The weight percentages of the four regions in the ACFB01 >100K sample

| | Peak region | | | |
|---|---|---|---|---|
| | P1 | P2 | P3 | P4 |
| Mw | 2.058 × 10⁷ | 1.012 × 10⁷ | 2.484 × 10⁶ (±0.280%) | 2.451 × 10⁵ |
| | (±1.042%) | (±0.300%) | (2484K) | (±0.705%) |
| | (20580K) | (10120K) | | (245.1K) |
| % | 1.71 | 14.54 | 23.21 | 60.54 |

### 3. Evaluation for Activity of Polysaccharide of Antrodia camphorata Fruiting Body, ACFB01

(1) Effect of ACFB01 on Dendritic Cell Maturation.

At present, it is known that TNF-α is an important indicator for dendritic cell maturation (Huang R Y, Yu Y L, Cheng W C, OuYang C N, Fu E, Chu C L. Immunosuppressive effect of quercetin on dendritic cell activation and function. J Immunol. 2010 Jun. 15; 184(12):6815-21). Therefore, mouse bone marrow cells were treated with the crude polysaccharide of Antrodia camphorata fruiting body, ACFB01 sample, with different doses of 2.5-20 μg/ml in this experiment, to confirm whether the crude polysaccharide of Antrodia camphorata fruiting body, ACFB01 sample, had an effect to mature dendritic cells.

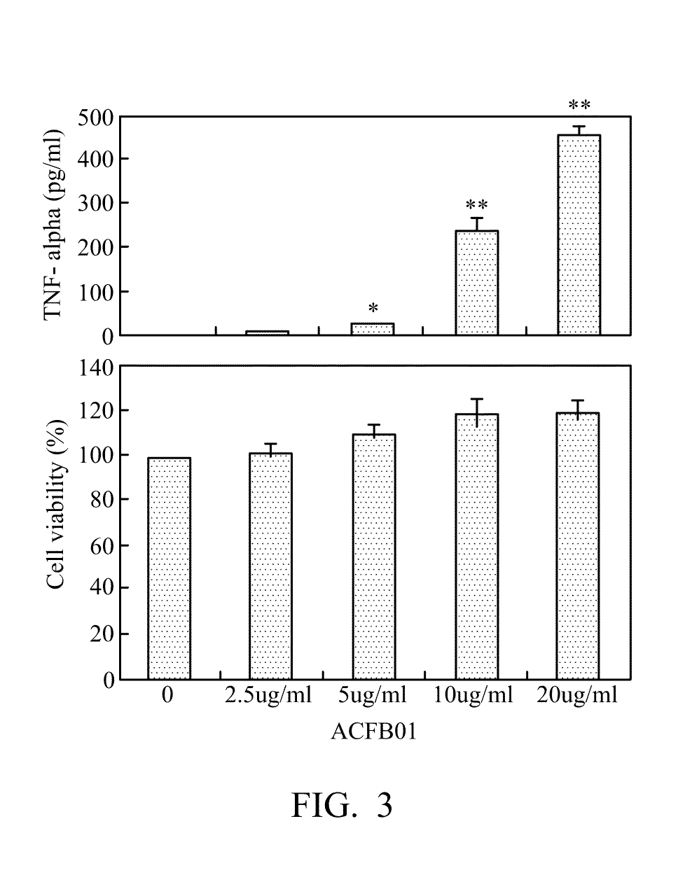

After mouse bone marrow-derived dendritic cells (BMDCs) were treated with the crude polysaccharide of Antrodia camphorata fruiting body, ACFB01 sample, with different dose for 4 hours, cell culture medium from each treatment group was collected and an enzyme-linked immunosorbent assay (ELISA) was performed thereto to determine the content of TNF-α secreted by each treatment group, and the cell viability for cells of each treatment group was also determined. The results are shown in FIG. 3. In FIG. 3, the value shown therein is a mean value for three different wells of each treatment group determined by each time, and the standard deviation is marked on the top of each bar. * and ** mean that there is a significant difference between the value shown and that of the no treatment control group (*p<0.05; **p<0.01, student t-test).

From the results it is found that ACFB01 has the effect of stimulating TNF-α secretion, and that shows dose-dependent relationship (FIG. 3), and this result represents that ACFB01 has the ability to mature dendritic cells. In addition, this result also shows that ACFB01 in the effective dose range does not decrease the cell viability of the dendritic cells, on the contrary, it resulted in a slight increment for cell numbers (FIG. 3).

(2) Effect of Fractions with Different Molecular Weights in the ACFB01 Sample on Dendritic Cell Maturation.

Purification and isolation were further performed on the polysaccharide, ACFB01, and the polysaccharide, ACFB01, was separated into different fractions by molecular weight difference. The different fractions were tested on mouse bone marrow cells.

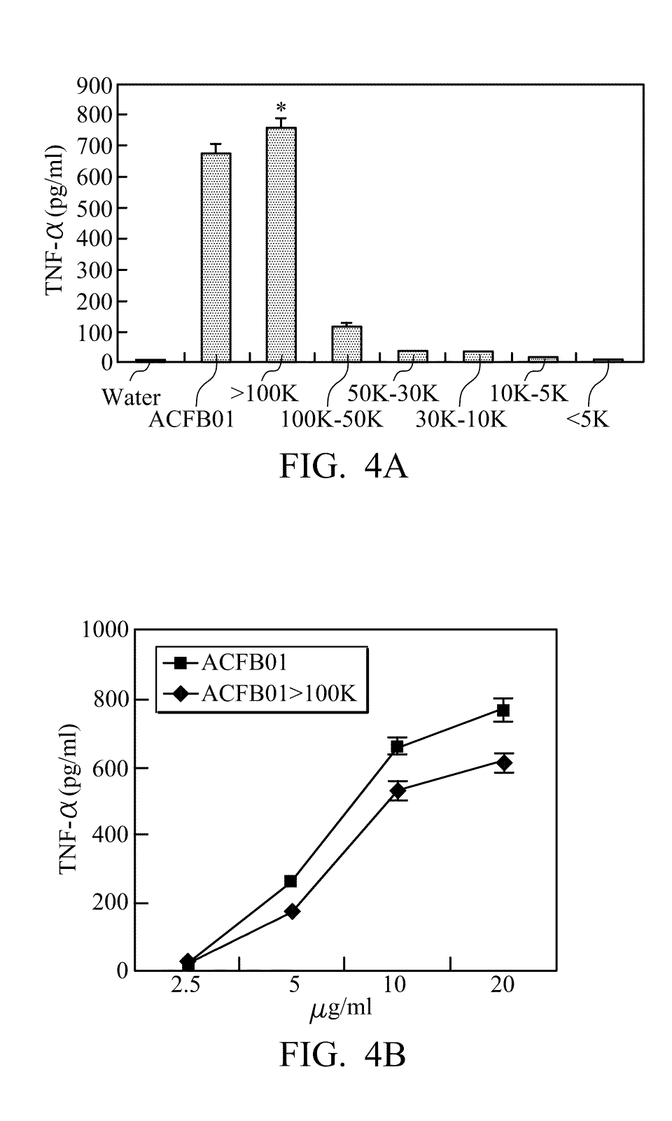

After mouse bone marrow-derived dendritic cells (BMDCs) were treated with fractions with different molecular weights of ACFB01 sample (20 μg/ml) for 4 hours, a cell culture medium from each treatment group was collected and an enzyme-linked immunosorbent assay (ELISA) was performed thereto to determine the content of TNF-α secreted by each treatment group. The results are shown in FIG. 4A. In FIG. 4A, the value shown therein is a mean value for three different wells of each group determined by each time, and the standard deviation is marked on the top of each bar. * means that there is a significant difference between the value shown and that of the ACFB01 treatment group (*p<0.05, student t-test). The results show that the part of ACFB01, which is capable of activating the maturation of dendritic cells, is the fraction of which the molecular weight is greater than 100 K Da (10 μg/ml).

After mouse bone marrow-derived dendritic cells (BMDCs) were treated with the fraction of which the molecular weight is greater than 100 K Da, of ACFB01, with different doses for 4 hours, a cell culture medium from each treatment group was collected and an enzyme-linked immunosorbent assay (ELISA) was performed thereon to determine the content of TNF-α secreted by each treatment group. The results are shown in FIG. 4B. In FIG. 4B, the value shown therein is a mean value for three different wells of each group determined by each time, and the standard deviation is marked on each point. * means that there is a significant difference between the value shown and that of the polysaccharide of Antrodia camphorata fruiting body (ACFB01) treatment group (*p<0.05, student t-test). The results show that the fraction of ACFB01, of which the molecular weight is greater than 100 K Da (ACFB01>100 K) has the effect of stimulating TNF-α secretion, and that also shows a dose-dependent relationship (FIG. 4B).

### 4. Evaluation for Activity of Polysaccharide of Antrodia camphorata Fruiting Body, ACFB01>100 K

(1) Ability of Polysaccharide of Antrodia camphorata Fruiting Body, ACFB01>100 K for Stimulating Mouse Bone Marrow Cells to Secrete Cytokines.

Ability of polysaccharide of Antrodia camphorata fruiting body, ACFB01>100 K, for stimulating mouse bone marrow cells to secrete cytokines was further analyzed by enzyme-linked immunosorbent assay (ELISA).

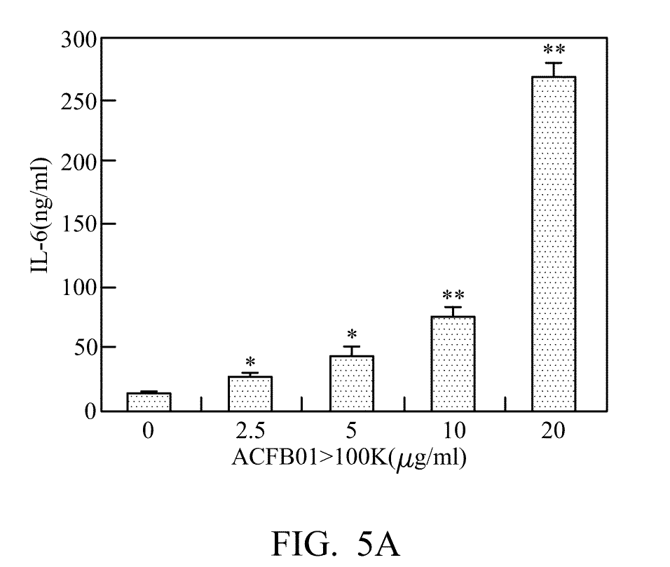

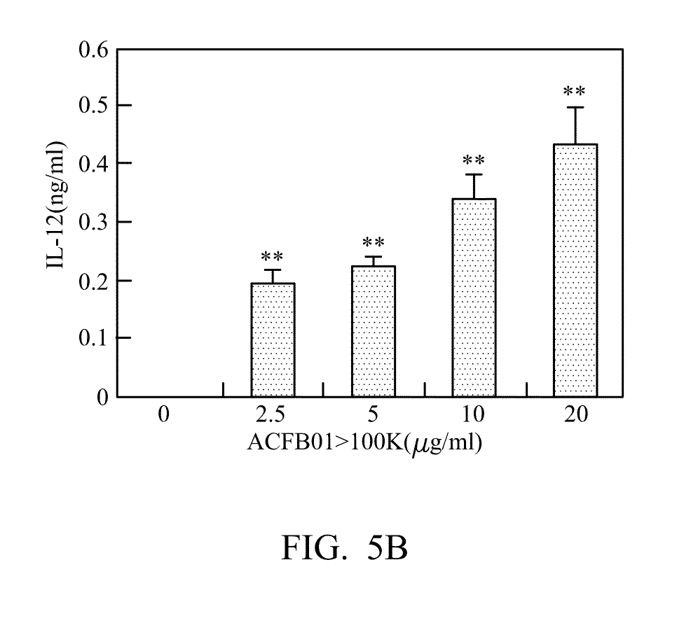

After mouse bone marrow-derived dendritic cells (BMDCs) were treated with the ACFB01>100 K sample with different doses (0-20 μg/ml) for 24 hours, a cell culture medium from each treatment group was collected and an enzyme-linked immunosorbent assay (ELISA) was performed thereon to determine the levels of IL-6 and IL-12 secreted by each treatment group. The results are shown in FIGS. 5A and 5B, respectively. In FIGS. 5A and 5B, the value shown is a mean value for three different wells of each group determined by each time, and the standard deviation is marked on the top of each bar. * and ** mean that there is a significant difference between the value shown and that of the no treatment control group (*p<0.05; **p<0.01, student t-test).

The results show that the polysaccharide of Antrodia camphorata fruiting body (ACFB01>100 K) is capable of increasing expression for IL-6 and IL-12 secretion and that showed a dose-dependent relationship (See FIGS. 5A and 5B, respectively). Since IL-12 is an important cytokine for activating Th1 cells and a Th1 cell has been known as a main cell for activating cytotoxic CD8⁺ T cells (Trinchieri G. Interleukin-12 and the regulation of innate resistance and adaptive immunity. Nat Rev Immunol. 2003 February; 3(2):133-46. Review), the polysaccharide of Antrodia camphorata fruiting body (ACFB01>100 K) has the potential for being used as an anti-cancer or anti-infection vaccine adjuvant.

(2) Ability of the Polysaccharide of Antrodia camphorata Fruiting Body, ACFB01>100 K for stimulating mouse bone marrow cells to secrete chemokines.

The ability of polysaccharide of Antrodia camphorata fruiting body, ACFB01>100 K, for stimulating mouse bone marrow cells to secrete chemokines was analyzed by enzyme-linked immunosorbent assay (ELISA).

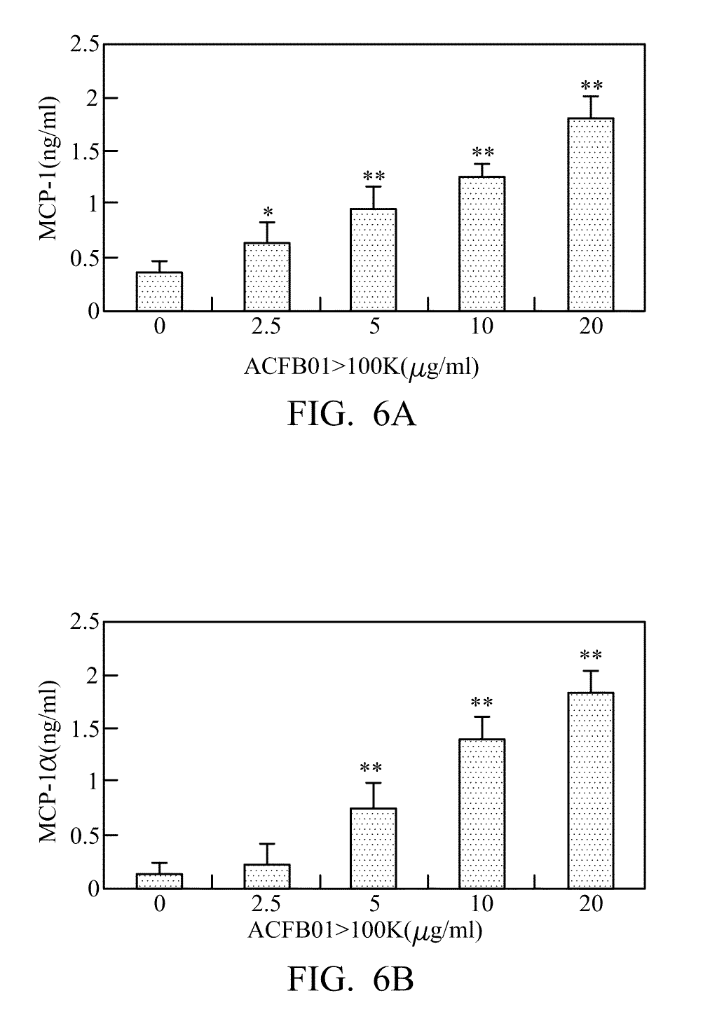

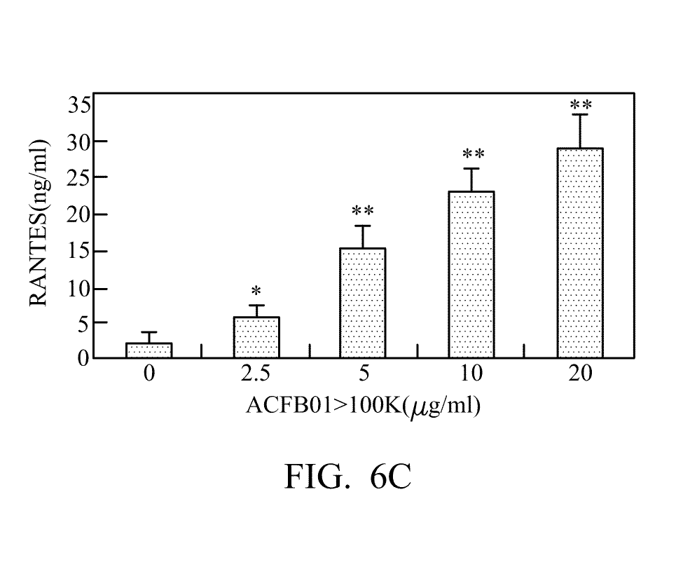

After mouse bone marrow-derived dendritic cells (BMDCs) were treated with the ACFB01>100 K sample with different doses (0-20 μg/ml) for 24 hours, a cell culture medium from each treatment group was collected and an enzyme-linked immunosorbent assay (ELISA) was performed thereon to determine the levels of MCP-1, MIP-1α and RANTES secreted by each treatment group. The results are shown in FIGS. 6A, 6B and 6C, respectively. In FIGS. 6A, 6B and 6C, the value shown is a mean value for three different wells of each group determined by each time, and the standard deviation is marked on the top of each bar. * and ** mean that there is a significant difference between the value shown and that of the no treatment control group (*p<0.05; **p<0.01, student t-test).

The results show that the ACFB01>100 K sample is capable of stimulating secretion of MCP-1, MIP-1α and RANTES and that shows a dose-dependent relationship (See FIGS. 6A, 6B and 6C). According to the results, it is known that the ACFB01>100 K sample not only promotes the maturation of dendritic cells, but also relates to the start of inflammation and adaptive immunity caused by dendritic cells.

(3) Ability of ACFB01>100 K for Stimulating Mouse Bone Marrow Cells to Express Surface Costimulators.

Ability of ACFB01>100 K sample for stimulating mouse bone marrow cells to express surface costimulators was analyzed by flow cytometer.

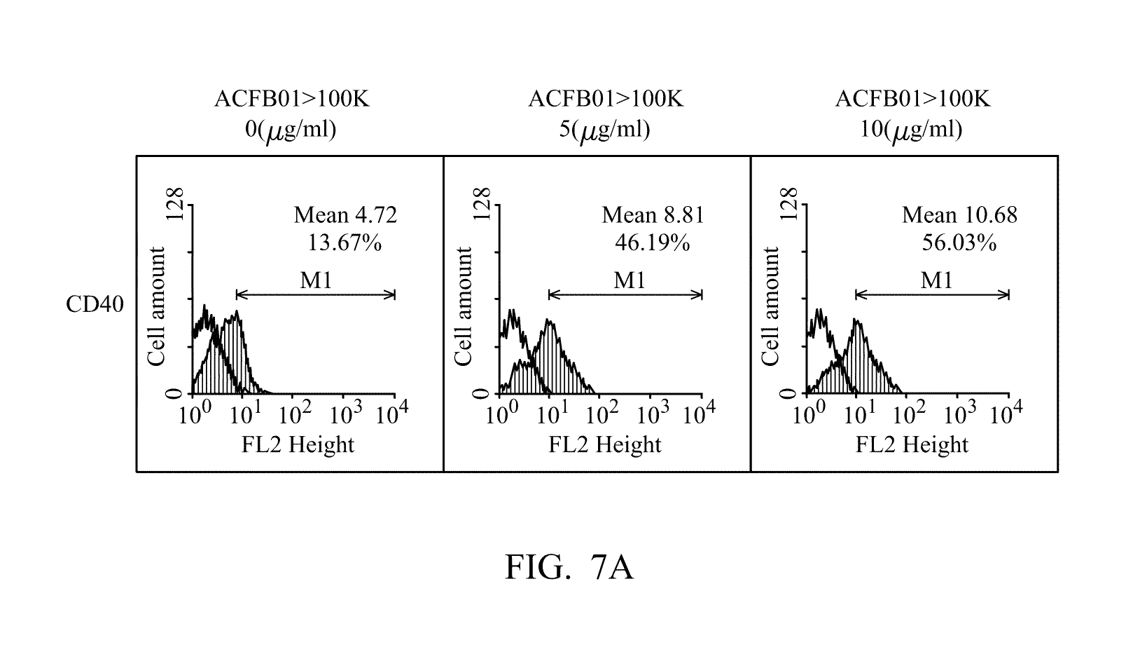

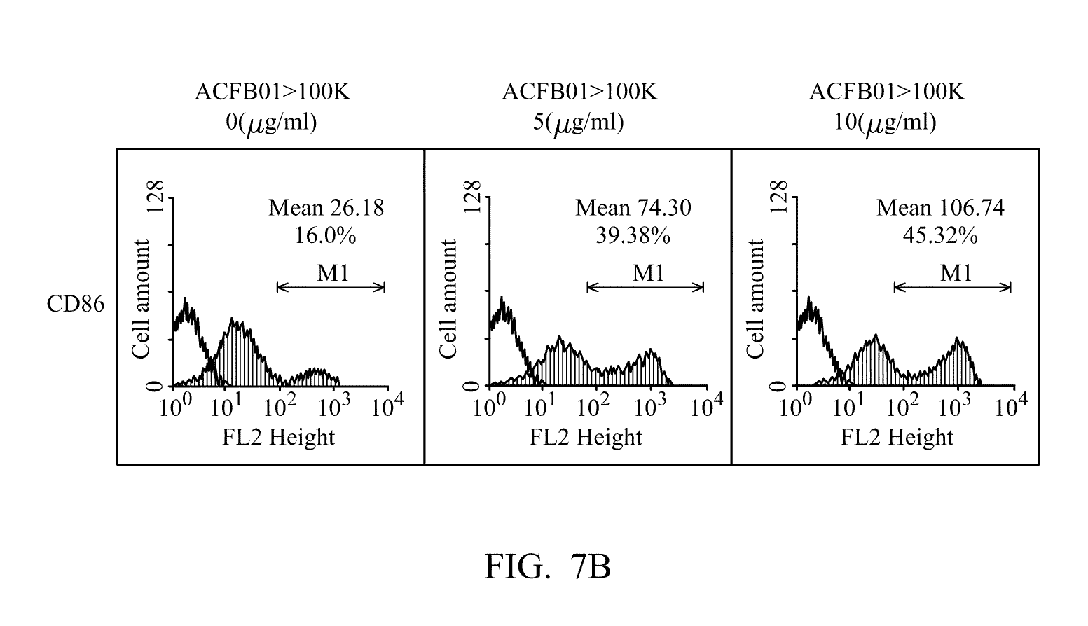

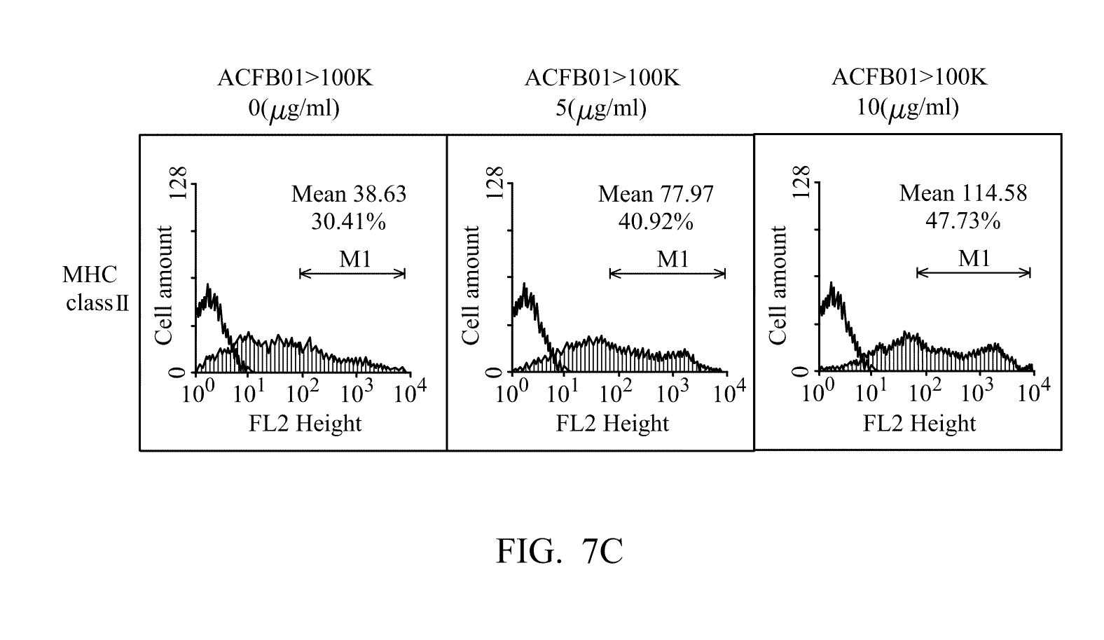

After mouse bone marrow-derived dendritic cells (BMDCs) were treated with the ACFB01>100 K sample (20 μg/ml) for 24 hours, the cells were collected. After that the cells were stained with specific antibodies and analyzed by flow cytometer. The results are shown in FIGS. 7A, 7B and 7C.

In FIGS. 7A, 7B and 7C, open histograms represent the background value which resulted from an isotype control antibody staining. Filled histograms represent the experimental groups of cells treated with ACFB01>100 K sample. Mean represents mean fluorescence intensity for all cells in that experiment. % represents percentage of total number of cells in the gate in total number of all analyzed cells.

The results show that ACFB01>100 K sample (20 μg/ml) is capable of stimulating expressions of surface costimulators, such as CD40 (FIG. 7A), CD86 (FIG. 7B) and MHC class II (FIG. 7C), similarly. According to the results, it is known that the ACFB01>100 K sample is indeed promoting the maturation of dendritic cells.

(4) Effect of the Polysaccharide of Antrodia camphorata Fruiting Body (ACFB01>100 K) on Mouse Bone Marrow-Derived Dendritic Cells (BMDCs) Inducing Activation of Antigen-Specific T Cells In Vitro.

The ability of mouse bone marrow-derived dendritic cells (BMDCs) treated with ACFB01>100 K sample for promoting activation of antigen specific T cells was determined in vitro.

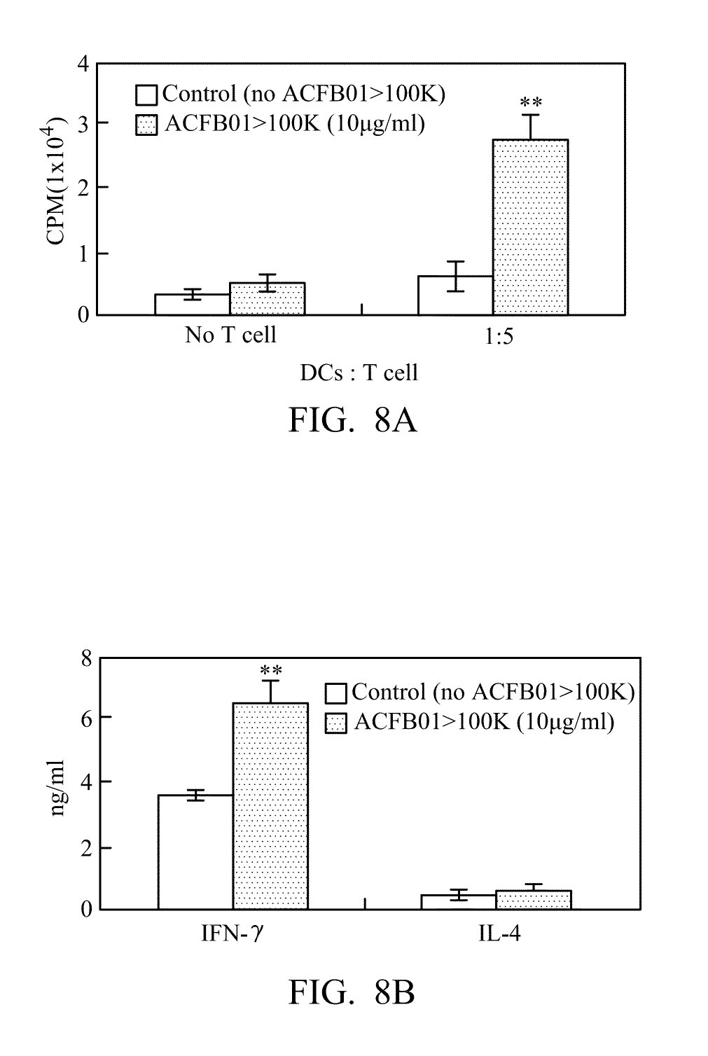

Dendritic cells (1×10⁶ cells/ml) were treated with or without ACFB01>100 K sample for 1 hour and then stimulated with OVA₂₅₇₋₂₆₄ peptide (2 ug/mL). After 16 hours, the cell culture medium was removed, and the dendritic cells were co-cultured with T cells taken from OT-I transgenic mouse (the ratio of the dendritic cells and the T cells was 1:5; control group: no T cell was provided) for 3 days. 18 hours before collecting the cell culture medium, [³H] thymidine was added into the cell culture medium. After that, cells were collected and the expression level of [³H] thymidine was determined to calculate the proliferation of the cells (Lin C C, Yu Y L, Shih C C, Liu K J, Ou K L, Hong L Z, Chen J D, Chu C L. A novel adjuvant Ling Zhi-8 enhances the efficacy of DNA cancer vaccine by activating dendritic cells. Cancer Immunol Immunother. 2011 July; 60(7):1019-27), and the results are shown in FIG. 8A. In addition, at the same time, the cell culture medium from each group was collected and an enzyme-linked immunosorbent assay (ELISA) was performed thereon to determine the expression levels of Interferon-γ/IFN-γ and IL-4 in the cell culture medium of each group. The results are shown in FIG. 8B. In FIGS. 8A and 8B, the value shown therein is a mean value for three different wells of each group determined by each time, and the standard deviation is marked on the top of each bar. ** means that there is a significant difference between the value shown and that of the control group (**p<0.01, student t-test). For the experiment mentioned above, three independent experiments were performed, and the three results therefrom showed repetition. FIGS. 8A and 8B show values from one of the three results.

According to FIG. 8A, it is known that the polysaccharide of Antrodia camphorata fruiting body (ACFB01>100 K) is capable of promoting dendritic cells to activate OVA specific T cells. Furthermore, according to FIG. 8B, it is known that the polysaccharide of Antrodia camphorata fruiting body (ACFB01>100 K) promotes dendritic cells to activate antigen-specific T cells through IFN-γ (Th1 response) pathway.

(5) Effect of the Polysaccharide of Antrodia camphorata Fruiting Body (ACFB01>100 K) on In Vivo Induction of Antigen-Specific T Cells Activation.

Ability of ACFB01>100 K sample for promoting activation of antigen-specific T cells was determined in vivo.

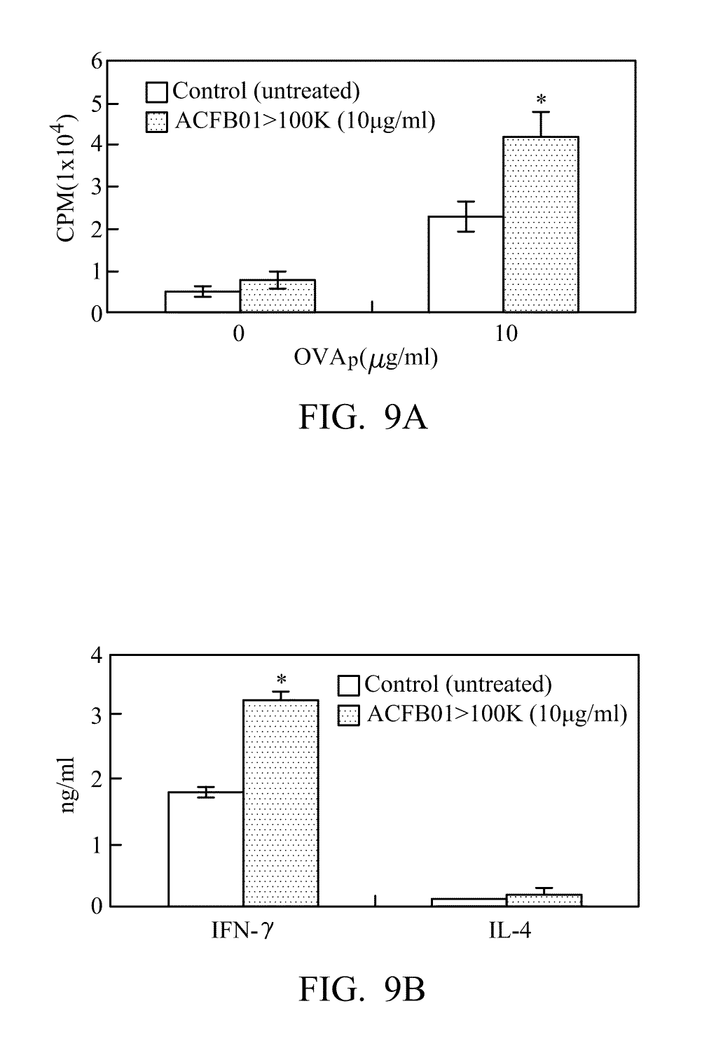

OT-I mice were divided into 4 groups: a group that received no treatment (control group), a group injected only with OVA₂₅₇₋₂₆₄ peptide to the paw, a group injected with OVA₂₅₇₋₂₆₄ peptide mixed with ACFB01>100 K sample into the paw, and a group injected only with ACFB01>100 K sample to the paw. There were three mice in each group. 10 days after the treatments, cells from a thigh lymph node of the mice from each group were mixed with immune dendritic cells isolated from bone marrow of normal C57BL/6 mice, and then stimulated with OVA peptide for 72 hours. 18 hours before collecting the cell culture medium, [³H] thymidine was added into the cell culture medium. After that, cells were collected and the expression level of [³H] thymidine was determined to calculate proliferation of the cells. The results are shown in FIG. 9A. In addition, at the same time, the cell culture medium from each group was collected and an enzyme-linked immunosorbent assay (ELISA) was performed thereto to determine the expression levels of Interferon-γ/IFN-γ and IL-4 in the cell culture medium of each group. The results are shown in FIG. 9B. In FIGS. 9A and 9B, the value shown therein is a mean value for three different wells of each group determined by each time, and the standard deviation is marked on the top of each bar. * means that there is a significant difference between the value shown and that of the control group (*p<0.05, student t-test). For the experiment mentioned above, three independent experiments were performed, and the three results therefrom showed repetition. FIGS. 9A and 9B show values from one of the three results.

The results show that the polysaccharide of Antrodia camphorata fruiting body (ACFB01>100 K) is indeed capable of promoting dendritic cells to activate OVA specific T cells, and the polysaccharide of Antrodia camphorata fruiting body (ACFB01>100 K) promoting dendritic cells to activate antigen-specific T cells is mainly through the IFN-γ (Th1 response) pathway.

(6) Effect of the Polysaccharide of Antrodia camphorata Fruiting Body (ACFB01>100 K) Combined with HER-2/Neu DNA Vaccine on Inhibition of Tumor.

It is known that HER-2/neu is an oncogene, which is capable of translating a 185 K Da transmembrane protein, and it has been proved that HER-2/neu is expressed in many kinds of tumors and is related to drug resistance. In order to evaluate the anti-tumor effect of the polysaccharide of Antrodia camphorata fruiting body (ACFB01>100 K) and DNA vaccine, HER-2/neu DNA vaccine (HER-2/neu (amino acid 1-650) of extracellular region used as carried antigen) was used as the DNA vaccine in this experiment (Lin C C, Chou C W, Shiau A L, Tu C F, Ko T M, Chen Y L, Yang B C, Tao M H, Lai M D. Therapeutic HER2/Neu DNA vaccine inhibits mouse tumor naturally overexpressing endogenous neu. Mol Ther. 2004 August; 10(2):290-301).

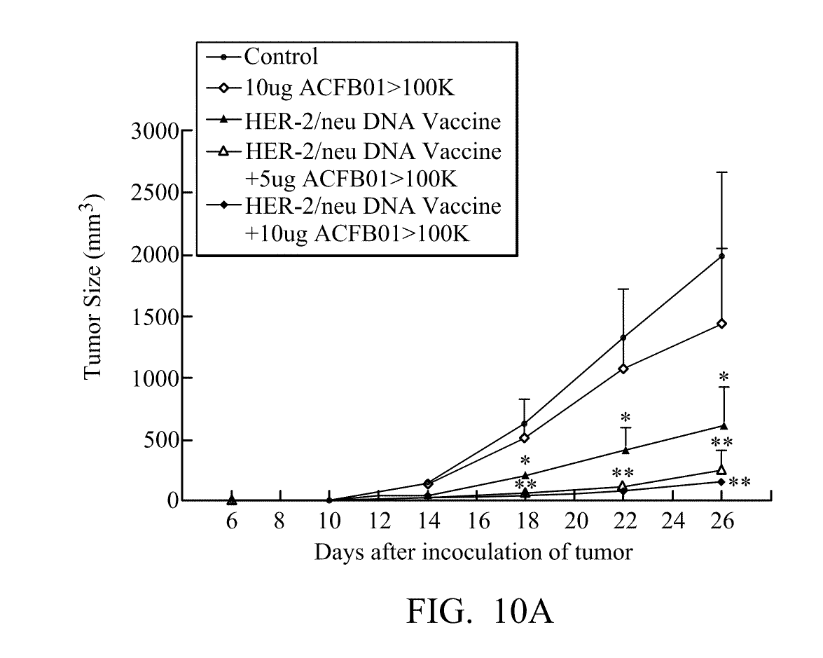

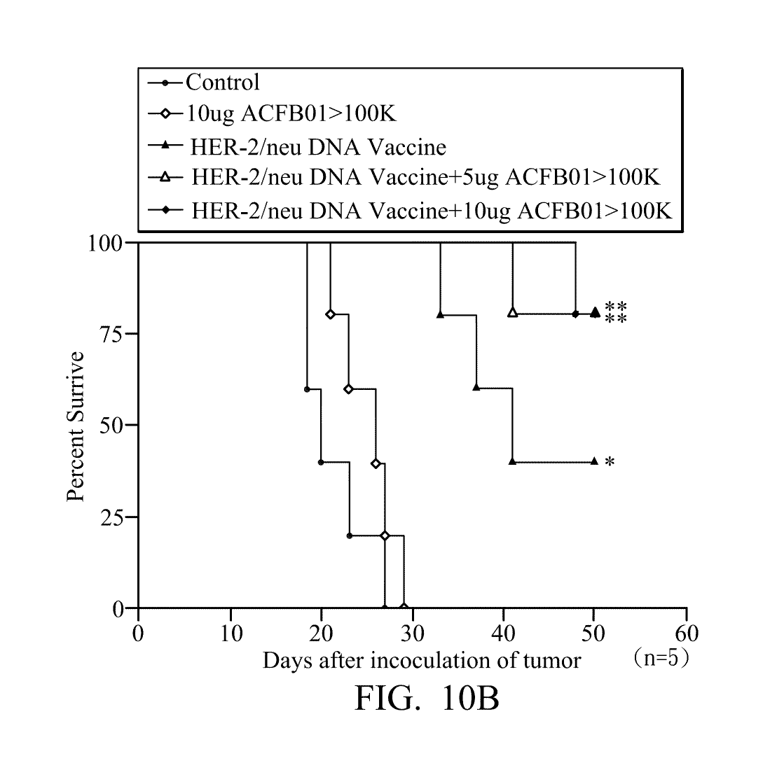

C3/HeN mice subcutaneously injected with MBT-2 tumor cells (bladder cancer cell overexpres sing HER-2/neu) were divided into five groups: a group that received no treatment (control group), a group injected only with 10 μg of ACFB01>100 K sample, a group injected only with HER-2/neu DNA vaccine, a group injected with HER-2/neu DNA mixed with 5 μg of ACFB01>100 K sample, and a group injected with HER-2/neu DNA mixed with 10 μg of ACFB01>100 K sample. According to tumor growth size and viability of mice, the level of tumor inhibition and life of the mice of each group were evaluated. Viability data were analyzed by Kaplan-Meier. The results are shown in FIGS. 10A and 10B. * mean that there is a significant difference between the value shown and that of the negative control group, and ** means that there is a significant difference between the value shown and that of the group injected with only HER-2/neu DNA vaccine. For the experiment mentioned above, two independent experiments were performed, 4 mice were used in each experiment, and the two results therefrom showed repetition.

The results show that as compared with the group injected with only HER-2/neu DNA vaccine, the group injected with HER-2/neu DNA vaccine mixed with 10 μg of ACFB01>100 K sample had the significant effect of inhibiting MBT-2 cell growth and increasing the viability of the mice with tumor.

(7) Effect of the Polysaccharide of Antrodia camphorata Fruiting Body (ACFB01>100 K) Combined with HER-2/Neu DNA Vaccine on Activation of Specific T Cells.

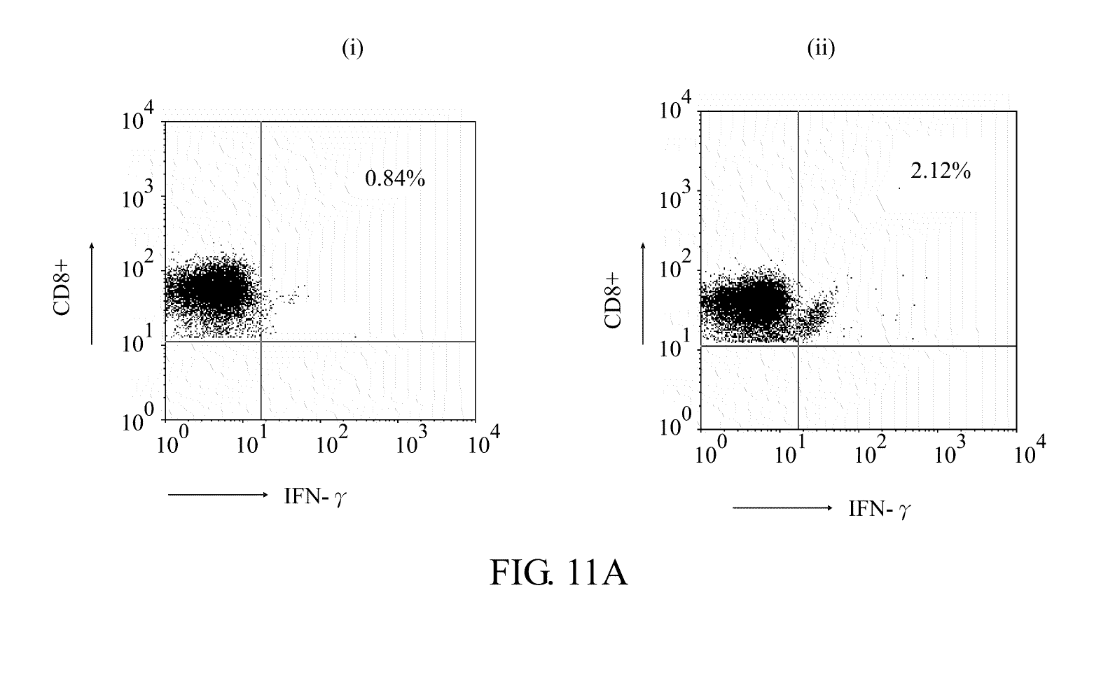

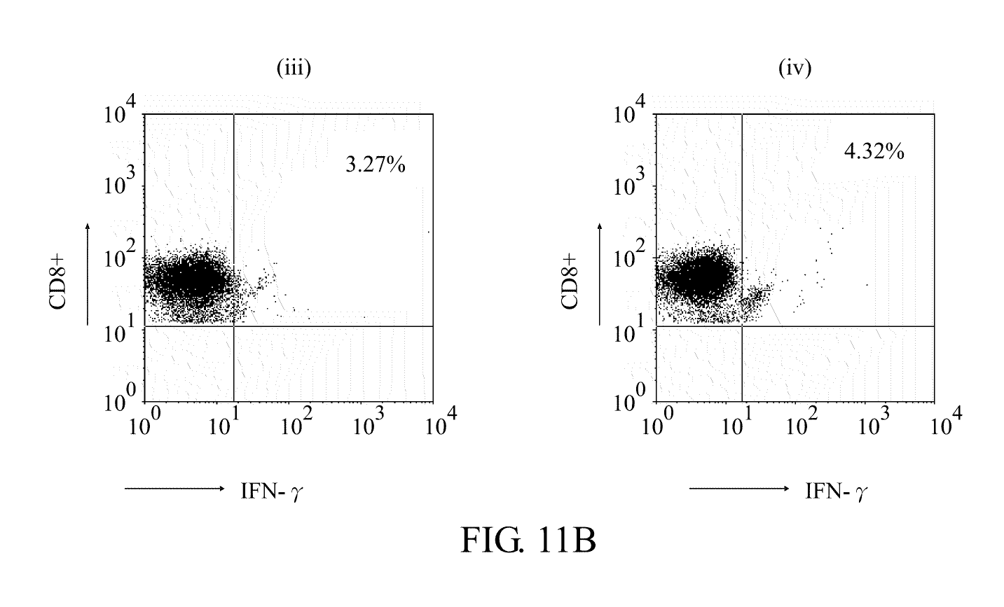

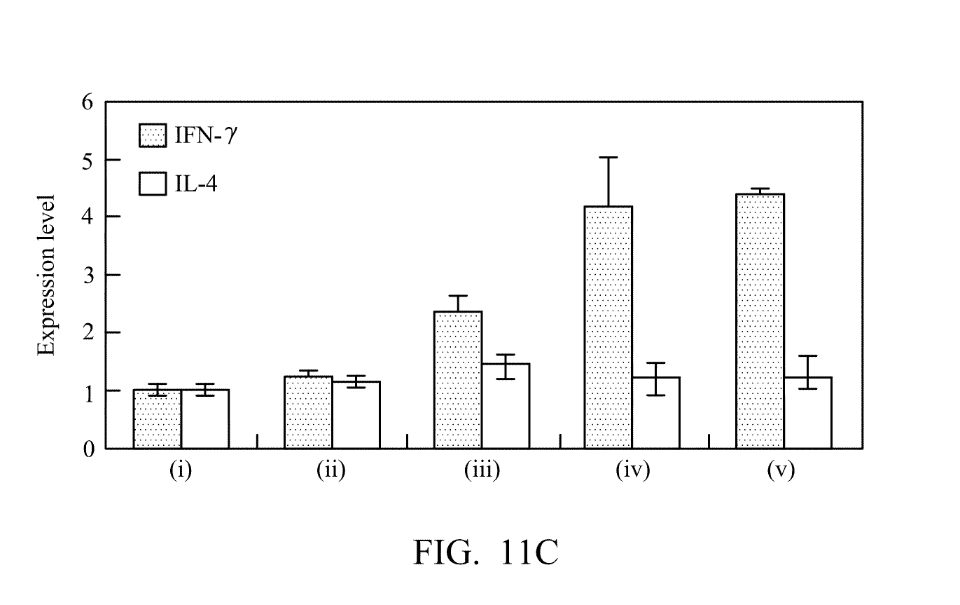

For the control group, the group injected only with HER-2/neu DNA vaccine, the group injected with HER-2/neu DNA mixed with 5 μg of ACFB01>100 K sample, and the group injected with HER-2/neu DNA mixed with 10 μg of ACFB01>100 K sample of the C3/HeN mice subcutaneously injected with MBT-2 tumor cells (bladder cancer cell overexpressing HER-2/neu) mentioned above, the percentages of HER-2/neu-specific CD8 positive cells secreting IFN-γ in total CD8 positive cells were determined by flow cytometer. The results are shown in FIGS. 11A and 11B. (i) the control group; (ii) the group injected only with HER-2/neu DNA vaccine; (iii) the group injected with HER-2/neu DNA mixed with 5 μg of ACFB01>100 K sample; and (iv) the group injected with HER-2/neu DNA mixed with 10 μg of ACFB01>100 K sample. For this experiment, two independent experiments were performed, and the two results therefrom were similar and showed repetition.

Moreover, CD8 positive cells of the control group, the group injected only with 10 μg of ACFB01>100 K sample, the group injected only with HER-2/neu DNA vaccine, the group injected with HER-2/neu DNA mixed with 5 μg of ACFB01>100 K sample, and the group injected with HER-2/neu DNA mixed with 10 μg of ACFB01>100 K sample of the C3/HeN mice subcutaneously injected with MBT-2 tumor cells mentioned above (bladder cancer cell overexpressing HER-2/neu) were isolated, and then real time quantitative RNA analysis was performed to the isolated CD8 positive cells to determine the expressions of IFN-γ and IL-4. The results are shown in FIG. 11C. (i) the control group; (ii) the group injected only with 10 μg of ACFB01>100 K sample; (iii) the group injected only with HER-2/neu DNA vaccine; (iv) the group injected with HER-2/neu DNA mixed with 5 μg of ACFB01>100 K sample; and (v) the group injected with HER-2/neu DNA mixed with 10 μg of ACFB01>100 K sample. In FIG. 11C, the values shown therein are mean values for IFN-γ and IL-4 expressions in 1×10⁵ cells of each treatment group as compared with those of the control group, and the standard deviation is marked on the top of each bar. * means that there is a significant difference between the value shown and that of the vehicle control group (*p<0.05, student t-test), and ** means that there is a significant difference between the value shown and that of the group injected only with HER-2/neu DNA vaccine (*p<0.05, student t-test).

The results show that in the mice with MBT-2 tumor, as compared with the treatment of injecting only HER-2/neu DNA vaccine, the treatment of injecting HER-2/neu DNA mixed with 5 μg of ACFB01>100 K sample is capable of increasing more IFN-γ positive CD8 positive T cells (FIGS. 11A and 11B), and bone marrow-derived dendritic cells inducing activation of antigen-specific is achieved through IFN-γ (FIG. 11C). Therefore, these investigative results confirm that ACFB01>100 K polysaccharide indeed has the potential to develop as an adjuvant for an anti-cancer DNA vaccine.

(8) Inhibiting Effect of DC Vaccine Pulsed with the Polysaccharide of Antrodia Camphorata Fruiting Body (ACFB01>100 K) Combined with Liver Cancer Cell Lysate to Tumor of Orthotopic Liver Cancer.

In order to evaluate the inhibiting effect of the polysaccharide of Antrodia camphorata fruiting body (ACFB01>100 K) to an orthotopic liver cancer, Balb/c mice livers into which was implanted mouse liver tumor cells (ML-1 tumor; 2×10⁶) were used as animal models for orthotopic liver cancer (Huang T T, Yen M C, Lin C C, Weng T Y, Chen Y L, Lin C M, Lai M D. Skin delivery of short hairpin RNA of indoleamine 2,3 dioxygenase induces antitumor immunity against orthotopic and metastatic liver cancer. Cancer Sci. 2011 December; 102(12):2214-20).

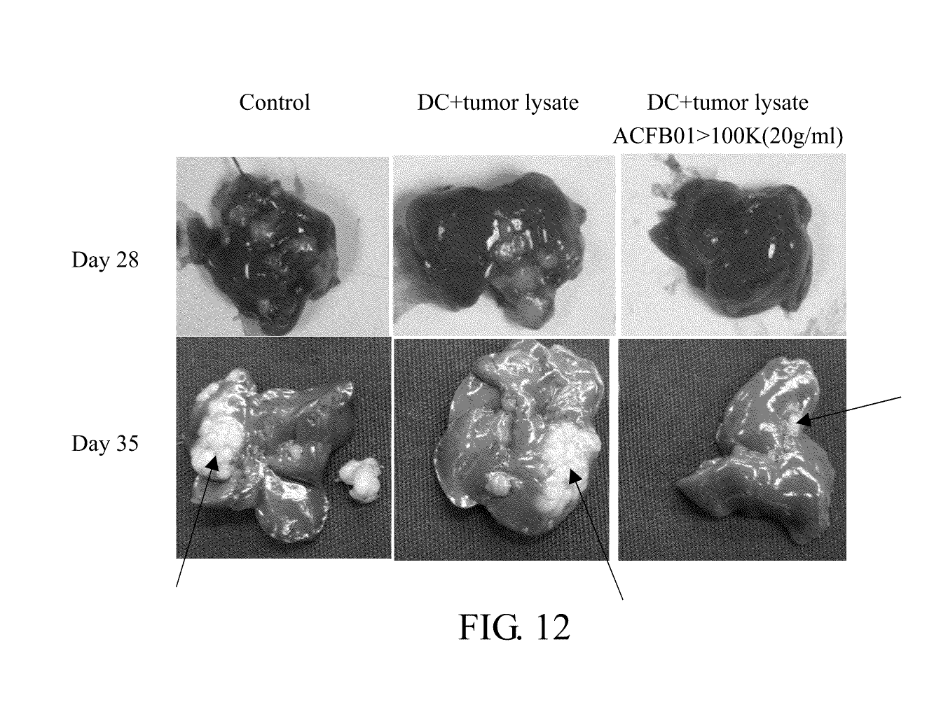

5 days after the mice were implanted with tumor cells, BALB/C mouse bone marrow-derived dendritic cells co-cultured with tumor lysate or with tumor lysate+ACFB01>100 K sample were further subcutaneously implanted into the mice with liver tumors. 28 days and 35 days after being implanted with tumor cells, the mice were sacrificed and liver taken to observe the condition of growth of cancer cells, and there were two mice in each round of the experiment. The results are shown in FIG. 12. Locations indicated by arrows represent cancer cells.

The results show that dendritic cell vaccine pulsed with the polysaccharide (ACFB01>100 K) combined with ML-1 tumor lysate has significant ability against tumor, as compared with dendritic cell vaccine only pulsed with ML-1 tumor lysate (FIG. 12). This result confirms that the polysaccharide of Antrodia camphorata fruiting body (ACFB01>100 K) is capable of enhancing the effect of dendritic cell vaccine against liver cancer, and has effect of adjuvant for anti-cancer dendritic cell vaccine.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A vaccine adjuvant, comprising:
a polysaccharide derived from Antrodia camphorata fruiting body,
wherein the molecular weight of the polysaccharide is greater than 100 K Da.

2. The vaccine adjuvant as claimed in claim 1, wherein the molecular weight of the polysaccharide is between 2.0×10⁵ Da and 2.1×10⁷ Da.

3. The vaccine adjuvant as claimed in claim 1, wherein the polysaccharide is obtained by an extraction process, and the extraction process comprises:
(a) adding powder of the Antrodia camphorata fruiting body into water to form a mixture;
(b) heating the mixture under reflux;
(c) after step (b), removing an insoluble matter from the mixture;
(d) after step (c), adding ethanol to the mixture to perform a precipitating step and obtain a precipitate; and
(e) performing an isolating step to the precipitate to obtain a fraction the molecular weight of which is greater than 100 K Da of the precipitate.

4. The vaccine adjuvant as claimed in claim 1, wherein the polysaccharide is capable of activating a dendritic cell.

5. The vaccine adjuvant as claimed in claim 1, wherein the polysaccharide is capable of enhancing a dendritic cell to express a major histocompatibility complex (MHC) class II, CD40 and/or CD86.

6. The vaccine adjuvant as claimed in claim 1, wherein the polysaccharide is capable of enhancing a dendritic cell to induce activation of an antigen-specific T cell.

7. The vaccine adjuvant as claimed in claim 1, wherein the polysaccharide is capable of enhancing T cell proliferation and/or expression of interferon which is a Th1 cell cytokine.

8. A vaccine composition, comprising:
the vaccine adjuvant as claimed in claim 1; and
an antigen or DNA encoding the antigen.

9. The vaccine composition as claimed in claim 8, wherein the molecular weight of the polysaccharide is between 2.0×10⁵ Da and 2.1×10⁷ Da.

10. The vaccine composition as claimed in claim 8, wherein the antigen comprises phage, phage composition, virus, virus composition, rickettsia, rickettsia composition, actinomyces, actinomyces composition, bacteria, bacteria composition, fungus, fungus composition, protozoan, protozoan composition, tumor tissue, tumor cell, tumor cell composition, tumor antigen protein, or tumor antigen peptide.

11. The vaccine composition as claimed in claim 8, wherein the vaccine composition comprises an anti-cancer vaccine composition, an anti-virus vaccine composition or an anti-bacteria vaccine composition.

12. The vaccine composition as claimed in claim 11, wherein the anti-cancer vaccine composition is used against bladder cancer, liver cancer, leukemia, colorectal cancer, breast cancer, kidney cancer, lung cancer, pancreatic cancer, prostate cancer, cervical caner, or head and neck cancer.

13. A method for preparing a vaccine adjuvant, comprising:
using a polysaccharide derived from Antrodia camphorata fruiting body.

14. The method for preparing a vaccine adjuvant as claimed in claim 13, wherein the molecular weight of the polysaccharide is between 2.0×10⁵ Da and 2.1×10⁷ Da.

15. The method for preparing a vaccine adjuvant as claimed in claim 13 wherein the polysaccharide is obtained by an extraction process, and the extraction process comprises:
(a) adding powder of the Antrodia camphorata fruiting body into water to form a mixture;
(b) heating the mixture under reflux;
(c) after step (b), removing an insoluble matter from the mixture;
(d) after step (c), adding ethanol to the mixture to perform a precipitating step and obtain a precipitate; and
(e) performing an isolating step to the precipitate to obtain a fraction the molecular weight of which is greater than 100 K Da of the precipitate.

16. The method for preparing a vaccine adjuvant as claimed in claim 13, wherein the polysaccharide is capable of activating a dendritic cell.

17. The method for preparing a vaccine adjuvant as claimed in claim 13, wherein the polysaccharide is capable of enhancing a dendritic cell to express a major histocompatibility complex (MHC) class II, CD40 and/or CD86.

18. The method for preparing a vaccine adjuvant as claimed in claim 13, wherein the polysaccharide is capable of enhancing a dendritic cell to induce activation of an antigen-specific T cell.

19. The method for preparing a vaccine adjuvant as claimed in claim 13, wherein the polysaccharide is capable of enhancing T cell proliferation and/or expression of interferon which is a Th1 cell cytokine.
